SEQUENCE LISTING (4 sequences)

SEQ ID NO: 1 - RNA, 22 nt, Homo sapiens
ucguaccgugaguaauaaugcg

SEQ ID NO: 2 - RNA, 23 nt, Homo sapiens
uuaaugcuaaucgugauaggggu

SEQ ID NO: 3 - RNA, 23 nt, Homo sapiens
uagcaccauuugaaaucaguguu

SEQ ID NO: 4 - RNA, 23 nt, Homo sapiens
caacggaaucccaaaagcagcug

(19) United States
(12) Patent Application Publication
(10) Pub. No.: US 2019/0390275 A1
(43) Pub. Date: Dec. 26, 2019

(54) CHRONIC KIDNEY DISEASE DIAGNOSTIC

(71) Applicant: University College Cardiff Consultants Limited, Cardiff (GB)

(72) Inventors: Timothy Bowen, Cardiff (GB); Donald Fraser, Cardiff (GB)

(21) Appl. No.: 16/069,668

(22) PCT Filed: Jan. 26, 2017
(86) PCT No.: PCT/GB2017/050195

§ 371 Date: Jul. 12, 2018

(30) Foreign Application Priority Data
Jan. 28, 2016 (GB) 1601544.8

(51) Int. Cl. C12Q 1/6883

(52) U.S. Cl. CPC C12Q 1/6883; C12Q 2600/178; C12Q 2600/106; C12Q 2600/118; C12Q 2600/112; C12Q 2600/158

(57) ABSTRACT

The invention relates to a diagnostic method for determining at least one of, or a combination of, the following: has-miR-155-5p, hsa-miR-126-3p and has-miR-29b-3p in a urine sample for determining the existence of, or progression of, chronic kidney disease in a subject; and a kit of parts for undertaking said method.

### FIELD OF THE INVENTION

The invention relates to a diagnostic method for determining at least one of, or a combination of, the following: has-miR-155-5p, hsa-miR-126-3p and has-miR-29b-3p in a urine sample for determining the existence of, or progression of, chronic kidney disease in a subject; and a kit of parts for undertaking said method.

### BACKGROUND OF THE INVENTION

Chronic Kidney Disease (CKD) is a progressive loss in renal function over a period of months or years. The most common recognised cause of CKD is diabetes mellitus; others include idiopathic (i.e. unknown cause, often associated with small/diminished kidneys on renal ultrasound), hypertension, and glomerulonephritis. Together, these cause about 75% of all adult cases. The symptoms of worsening kidney function are not specific, and might include feeling generally unwell and experiencing a reduced appetite but may also be identified when they lead to one of its recognized complications, such as cardiovascular disease, anaemia, or pericarditis.

CKD is differentiated from acute kidney disease in that the condition must be present for over 3 months. CKD is diagnosed by the presence of a glomerular filtration rate (GFR)<60 ml/min/1.73 m² for 3 months or by the presence of kidney damage for 3 months. Kidney damage is defined by structural or functional abnormalities of the kidney, with or without decreased GFR, manifest by either pathological abnormalities or by markers of kidney damage, including abnormalities in the composition of the blood or urine, or abnormalities in imaging tests. CKD is typically divided into five stages on the basis of the amount of residual excretory function:
i) Stage 1: Slightly diminished function; kidney damage with normal or relatively high GFR (90 ml/min/1.73 m²). Kidney damage is defined as pathological abnormalities or markers of damage, including abnormalities in blood or urine tests or imaging studies.
ii) Stage 2: Mild reduction in GFR (60-89 ml/min/1.73 m²) with kidney damage. Kidney damage is defined as pathological abnormalities or markers of damage, including abnormalities in blood or urine tests or imaging studies.
iii) Stage 3: Moderate reduction in GFR (30-59 ml/min/1.73 m²): British guidelines distinguish between stage 3A (GFR 45-59) and stage 3B (GFR 30-44) for purposes of screening and referral.
iv) Stage 4: Severe reduction in GFR (15-29 ml/min/1.73 m²)[1] Preparation for renal replacement therapy.
v) Stage 5: Established kidney failure (GFR<15 ml/min/1.73 m²), permanent renal replacement therapy, or end-stage kidney disease.

CKD is a major challenge to global health with serious implications for well being and economic output. The presence of CKD confers a markedly increased risk of cardiovascular disease which tends to be the most common cause of death amongst CKD patients rather than complete renal failure. There currently exists no definitive treatment plan for CKD patients, with the main treatment strategies aimed at ensuring any existing conditions, such as diabetes and high blood pressure, are carefully managed in addition to lifestyle changes including diet and exercise.

Apart from controlling other risk factors, the goal of therapy is to slow down or halt the progression of CKD to stage 5. Screening of at-risk people is therefore important because treatment options do exist that can delay the progression of CKD. For example, if an underlying cause of CKD, such as vasculitis, or obstructive nephropathy (blockage to the drainage system of the kidneys) is found, it may be treated directly to slow and prevent further damage.

However, the disease does not typically lead to the patient to feel unwell until the kidney damage is unfortunately at an advanced stage. A potential end-point for numerous causal mechanisms, CKD is frequently accompanied by proteinuria. However, the predictive value of current non-invasive prognostic indicators such as urine protein quantification is limited since changes only become apparent following disease onset. Further, the present diagnostic and prognostic test for intrinsic renal disease, a biopsy, has a 3% risk of major complications.

There is, thus, an unmet need for the identification of new CKD biomarkers for use in the diagnosis and prediction of the disease state.

Powerful RNA detection techniques are now available for analysis of body fluids, but endogenous RNase activity degrades large transcripts, such as mRNAs. Micro RNAs (miRNAs) are short, endogenous, single-stranded noncoding RNA transcripts that inhibit target gene expression by translational repression and/or mRNA degradation. There are a variety of processes regulated by miRNA such as embryonic development and alteration of miRNA expression has been linked to tumour progression. Extracellular miRNAs are present in most biological fluids and are relatively stable. miRNAs are selectively exported from cells using membrane-derived vesicles (exosomes and microparticles), lipoproteins, and other ribonucleoprotein complexes.

Currently more than 1000 human miRNAs have been identified. Many of these exhibit tissue-specific expression patterns, the dysregulation of which has been associated with various diseases. Quantitative analysis of miRNAs from body fluids therefore offers an alternative approach for the prognosis and/or diagnosis of various disorders including tissue injury, cardiovascular disease, autoimmune disease and cancer.

We consider the use of urinary biomarkers may provide a non-invasive, safe and cost-effective approach to obtaining important CKD diagnostic and prognostic information, and would be clearly attractive to clinical scientists and industry alike.

We disclose herein that increased levels of expression of at least one miRNA, or a specific subset of miRNAs, correlate with kidney disease and therefore the, or their, detection can be used as a non-invasive test for the determination of the existence of chronic kidney disease in a subject and also for monitoring disease progression. This useful link between miRNA expression and the staging of chronic kidney disease in a subject allows appropriate treatment plans and therapies to be stratified accordingly.

### STATEMENTS OF INVENTION

According to a first aspect of the invention there is provided a diagnostic method for determining chronic kidney disease in a human subject comprising detecting the level or amount of at least one of the following miRNAs selected from the group comprising hsa-miR-155-5p, hsa-miR-126-3p and hsa-miR-29b-3p or any combination thereof in an isolated urine sample from said human subject wherein an increased level(s) or amount(s) of said at least one miRNA(s) relative to the same miRNA in a control is indicative of chronic kidney disease.

In a preferred embodiment of the invention chronic kidney disease includes all stages i-v of chronic kidney disease and in particular stages iii-v i.e. the advance stages of kidney disease.

In yet a further preferred method of the invention said method detects hsa-miR-155-5p and/or hsa-miR-126-3p and/or hsa-miR-29b-3p mRNA or their respective cDNA.

In yet a further preferred method of the invention said method detects hsa-miR-155-5p and hsa-miR-126-3p mRNA; hsa-miR-155-5p and hsa-miR-29b-3p mRNA; hsa-miR-126-3p and hsa-miR-29b-3p mRNA; hsa-miR-155-5p and hsa-miR-126-3p and hsa-miR-29b-3p mRNA; or their respective cDNA.

In yet a further preferred method still, said method detects the level or amount of a combination of hsa-miR-155-5p and/or hsa-miR-126-3p and/or hsa-miR-29b-3p mRNA, or their respective cDNA, and the level or amount of said combination of miRNAs is compared to the level or amount of the same combination of miRNAs in a control sample.

In a preferred method of the invention said method comprises:
i) providing a urine sample taken from a subject to be analyzed;
ii) forming a reaction mixture comprising the urine sample and one or more nucleic acid probes complementary to all or a part of a nucleotide encoding at least one or any combination of hsa-miR-155-5p, hsa-miR-126-3p and hsa-miR-29b-3p;
iii) providing conditions to detect or amplify a nucleic acid molecule encoding at least one or any combination of hsa-miR-155-5p, hsa-miR-126-3p and hsa-miR-29b-3p, in said urine sample;
iv) quantifying the level or amount of at least one or any combination of hsa-miR-155-5p, hsa-miR-126-3p and hsa-miR-29b-3p, in said urine sample and comparing said level or amount to that in a control wherein an increase in the level or amount of at least one of hsa-miR-155-5p, hsa-miR-126-3p and hsa-miR-29b-3p, or any combination thereof, in said sample relative to said same miRNA, or said same combination thereof, in said control indicates chronic kidney disease in said subject.

Additionally, or alternatively, said method detects the level or amount of a combination of hsa-miR-155-5p and hsa-miR-126-3p and hsa-miR-29b-3p mRNA, or their respective cDNA, and the level or amount of said combination of miRNAs is compared to the level or amount of the same combination of miRNAs in a control sample.

Chronic kidney disease typically refers to more established forms of kidney disease, ideally characterised by a glomerular filtration rate of 60 ml/min/1.73 m² or less persisting for greater than 3 months or by the presence of kidney damage for 3 months. Kidney damage is defined by structural or functional abnormalities of the kidney, with or without decreased GFR, manifest by either pathological abnormalities or by markers of kidney damage, including abnormalities in the composition of the blood or urine, or abnormalities in imaging tests. In a further preferred embodiment, the disclosed method is particularly effective in the determination of advanced stage forms of chronic kidney disease (UK guideline CKD stages 3-5 inclusive) and therefore, the determination of increased amount of at least one of hsa-miR-155-5p, hsa-miR-126-3p and hsa-miR-29b-3p is seen in those samples from subjects with advanced forms i.e. UK guideline CKD stages 3-5 of the disease.

Reference herein to urine sample refers to said sample, or a processed derivative thereof, taken from a subject who has or is suspected of having chronic kidney disease. Indeed, further processing of the sample may be required for accurate and robust quantification of urinary miRNAs.

In a further preferred method of the invention said urine sample ideally comprises cells and/or exosomes and/or vesicles.

Ideally, a processed derivative of a urine sample is used such as, but not limited to, cells harvested from a urine sample and, if necessary, re-suspended in an alternative medium.

Reference herein to a control is, ideally, to a further sample of urine taken from an individual, or the mean of a group of individuals, not having or presenting with chronic kidney disease, in particular late stage (iii-v) chronic kidney disease.

As disclosed herein, it has unexpectedly been found that the expression levels or amounts of the miRNAs disclosed herein are elevated specifically in CKD, particularly late stage (iii-v) CKD, when compared to healthy individuals or those not having or presenting with CKD particularly late stage CKD, although they may be suffering from a disease, such as diabetes, that is likely eventually to give rise to kidney disease leading to CKD. This is an advantageous finding, as the method disclosed herein permits one to identify patients suffering from late stage CKD from those suffering from other diseases that may give rise to late stage CKD as well as healthy individuals; thus allowing the determination of clinical treatment.

Reference herein to hsa-miR-155-5p, hsa-miR-126-3p and hsa-miR-29b-3p refers to miRNAs as defined in the miRNA database by their accession numbers: MIMAT0000646; MIMAT0000445; and MIMAT0000100, respectively.

In a preferred method of the invention hsa-miR-126-3p, hsa-miR-155-5p, and hsa-miR-29b-3p comprises the nucleotide sequence set forth in SEQ ID NO: 1-3, specifically:

i)
(SEQ ID NO: 1)
5′-ucguaccgugaguaauaaugcg-3′;
ii)
(SEQ ID NO: 2)
5′-uuaaugcuaaucgugauaggggu-3′;
and
iii)
(SEQ ID NO: 3)
5′-uagcaccauuugaaaucaguguu-3′.

As will be appreciated by those skilled in the art, level(s) or amount(s) of the selected miRNAs can be identified using numerous detection techniques such as, but not limited to, nucleic acid detection including polymerase chain reaction [PCR] based methods, or the like. The detection methods described herein may be qualitative and/or quantitative. However, other conventional techniques, as will be appreciated by those skilled in the art, can be used in accordance with the invention.

In a preferred method of the invention said method is a PCR based method.

In a preferred method of the invention said PCR based method is Real Time [RT] PCR.

It will be apparent to those skilled in the art that techniques available for measuring RNA are well known and, indeed, routinely practised by those in the clinical diagnostics field. Such techniques may include reverse transcription of RNA to produce cDNA and an optional amplification step followed by the detection of the cDNA or a product thereof.

In an alternative embodiment of the invention the level or amount of miRNA(s) may be measured by real-time quantitative PCR.

In a preferred embodiment, as will be appreciated by those skilled in the art, detection is most ideally undertaken using target-specific nucleic acid oligonucleotide probes that can specifically and accurately detect, and preferably allow quantification of the level or amount of each respective miRNA.

As used herein, the term “probe(s)” describes an oligonucleotide that hybridizes under physiological or reaction conditions to at least one of hsa-miR-155-5p, hsa-miR-126-3p and hsa-miR-29b-3p, or their respective cDNA(s). Those skilled in the art will recognize that the exact length of the oligonucleotide and its degree of complementarity with its target will depend upon the specific target selected, including the sequence of the target and the particular bases which make up that sequence.

It is preferred that the oligonucleotide probe be constructed so as to bind selectively with its target under physiological/reaction conditions, i.e., to hybridize specifically, or substantially more, to the target sequence than to any other sequence in the target sample under physiological/reaction conditions.

In order to be sufficiently selective the oligonucleotide probe should comprise at least 7 and more preferably, at least 8, 9, 10, 11, 12, 13, 14 or 15, ideally consecutive, bases which are complementary to the specific miRNA.

In a preferred method of the invention said oligonucleotide probe(s) is/are designed to bind to at least one of, or any combination of, the nucleotide sequence(s) set forth in SEQ ID NOs: 1-3.

In further preferred methods of working the invention the level or amount of at least one of hsa-miR-155-5p, hsa-miR-126-3p and hsa-miR-29b-3p is determined having regard to a reference gene or protein (such as a housekeeping gene) or a gene/protein within a control sample or from the same sample. Alternatively still, the level or amount is determined having regard to an internal standard where a genetic construct, such as a plasmid, expressing a known quantity of reference gene/protein is used.

Reference herein to an increased level or amount, refers to an increased level or amount of nucleic acid of at least one of hsa-miR-155-5p, hsa-miR-126-3p and hsa-miR-29b-3p in said urine sample compared to the level or amount of same in a control urine sample taken from a healthy individual or an individual not having or presenting with CKD, particularly late stage (iii-v) CKD. In all cases the normal or increased level or amount is statistically relevant at the 5% level or less.

Reference herein to an increased level or amount of a combination of said miRNAs, refers to the level or amount of a combination of hsa-miR-155-5p and/or hsa-miR-126-3p and/or hsa-miR-29b-3p mRNA, or their respective cDNA or a product thereof, when compared to the level or amount of said same combination of miRNAs in a control sample.

In a further preferred method of the invention the level or amount of expression of a given miRNA is normalised having regard to the expression of a reference miRNA such as, but not limited to, hsa-miR-191-5p.

Thus, increased expression refers to an increase in expression of a selected miRNA having regard to the expression of the same miRNA in control sample (for example, that taken from a healthy subject or a subject not having or presenting with CKD particularly late stage (iii-v) CKD) when normalised against hsa-miR-191-5p.

Reference herein to hsa-miR-191-5p refers to the miRNA as defined in the miRNA database by its accession number MIMAT0000440.

In a preferred method of the invention hsa-miR-191-5p comprises the nucleotide sequence set forth in SEQ ID NO: 4, specifically:

i)
(SEQ ID NO: 4)
5′-caacggaaucccaaaagcagcug-3′.

We have unexpectedly found that hsa-miR-155-5p, hsa-miR-126-3p and hsa-miR-29b-3p show an increased expression in advanced stages of kidney disease. Therefore, increased expression of at least one of hsa-miR-155-5p, hsa-miR-126-3p and hsa-miR-29b-3p, or any combination thereof, is indicative of a more severe form of disease showing reduced kidney function and with poorer predicted overall survival. By testing for the level or amount of expression of these markers one can determine with greater accuracy the stage of the disease with the aim of determining a suitable clinical course or treatment.

In a preferred embodiment of the invention, at least one of hsa-miR-155-5p, hsa-miR-126-3p and hsa-miR-29b-3p is of a level or amount that is at least 1.5-fold higher than the corresponding expression in a control sample taken from a healthy individual or an individual not having or presenting with CKD, particularly late stage (iii-v) CKD. More ideally at least one of hsa-miR-155-5p, hsa-miR-126-3p and hsa-miR-29b-3p is expressed at a level or amount that is between 1.5-30.0-fold higher, or at least 1.5-6.0-fold higher than the corresponding expression in a control sample taken from a healthy individual or an individual not having or presenting with CKD, particularly late stage (iii-v) CKD. More ideally still, at least one of hsa-miR-155-5p, hsa-miR-126-3p and hsa-miR-29b-3p is of a level or amount selected from the group comprising: 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, and 6.0 fold higher than the corresponding expression in a control sample taken from a healthy individual or an individual not having or presenting with CKD, particularly late stage (iii-v) CKD.

According to a further aspect of the invention, there is provided a method for monitoring the progression of chronic kidney disease in a human subject comprising repeating one or more of the afore, and ideally the same, method(s) periodically.

Ideally, different levels or amounts of said miRNA(s) are correlated with known chronic kidney disease staging techniques such that a simple in vitro assay can be used to reliably inform a clinician about, not only the existence of a chronic kidney disease, but also its stage or progression.

As will be appreciated by those skilled in the art, in the above method of the invention the relative change in the level or amount of expression can be used to assess how effective a treatment regimen is working, for example, by assaying the level or amount of a selected one or more miRNA(s) during the course of a given therapy to determine if there is a change in the miRNA level or amount, indicative of expression, in response to said treatment.

Accordingly, there is also provided a method for selecting a course of treatment comprising performing any one of the afore methods and then, depending upon the outcome of the method, determining a suitable or selected course of treatment.

Additionally, or alternatively, there is provided a method for treating chronic kidney disease comprising performing any one of the afore methods and then, depending upon the outcome of the method, undertaking a suitable or selected course of treatment.

According to a further aspect of the invention, there is provided a kit for performing any one or more of aforementioned methods wherein said kit comprises:
(a) at least one oligonucleotide probe(s) for detecting and quantifying the level or amount of at least one of the following hsa-miR-155-5p, miR-hsa-miR-126-3p and hsa-miR-29b-3p in a urine sample; and
(b) optionally, reagents and instructions pertaining to the use of said oligonucleotide(s).

Ideally the kit comprises a plurality of said probes for identifying a plurality, including any combination of, hsa-miR-155-5p, miR-hsa-miR-126-3p and hsa-miR-29b-3p in a urine sample.

Ideally, in the above aspect the instructions show how to determine the amount of said miRNA(s) and the conclusions to be drawn in view of their relative amounts.

In a further preferred kit of the invention said kit further comprises at least one oligonucleotide probe(s) for detecting and quantifying a reference miRNA such as, but not limited to, hsa-miR-191-5p

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

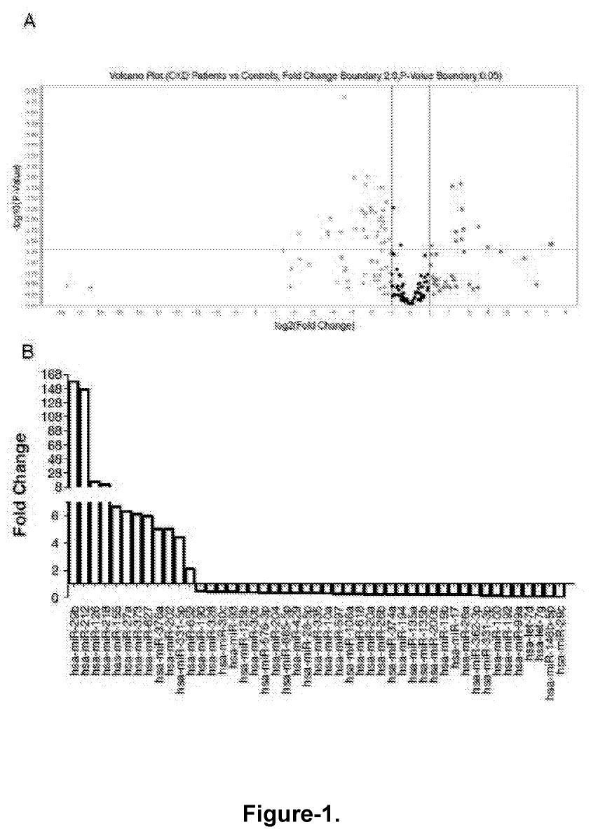

FIG. 1: Comparison of miRNA expression profiles in pooled urine samples from diabetic nephropathy patients and controls using TaqMan Low Density Array (TLDA) Human microRNA Card A. (A) Volcano plot showing fold-change and statistical significance to compare expression profiles of 377 urinary miRNAs between diabetic nephropathy patients (n=20; four pools of five patients) and unaffected control subjects (n=20; four pools of five control subjects). The negative log₁₀, of the p value is plotted on the y-axis, while the x-axis shows log₂ of the fold change in expression between the two experimental groups. The horizontal blue line represents a p value boundary of 0.05. (B) Fold change of miRNA expression between patients and controls. DataAssist™ Software (Life Technologies) was used to perform relative quantification for sample comparison, to perform t-test sample group comparisons, and to produce the above graphic output;

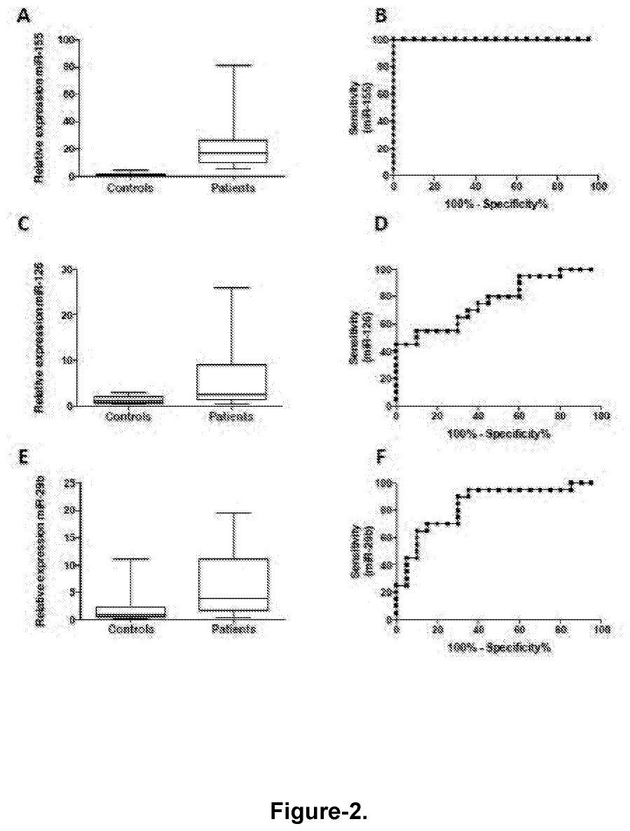

FIG. 2: Replication of significantly increased detection of selected microRNAs in individual urine samples. RT-qPCR analysis shows significant differences in detection of (A) hsa-miR-155-5p (p=0.0002), (C) hsa-miR-126-3p (p=0.0087) and (E) hsa-miR-29b-3p (p=0.0024) between the 20 patient and 20 control component urine samples from the pools used for the TLDA miRNA expression profiling shown in FIG. 1. Receiver operating characteristic curves are shown for (B) hsa-miR-155-5p (area under the curve (AUC)=1, p<0.0001), (D) hsa-miR-126-3p (AUC=0.77, p=0.003) and (F) hsa-miR-29b-3p (AUC=0.84, p=0.0002). Analysis was carried out by unpaired t-test with Welch's correction. Data were normalized to endogenous control hsa-miR-191-5p and, where appropriate, are presented as mean+/−SEM;

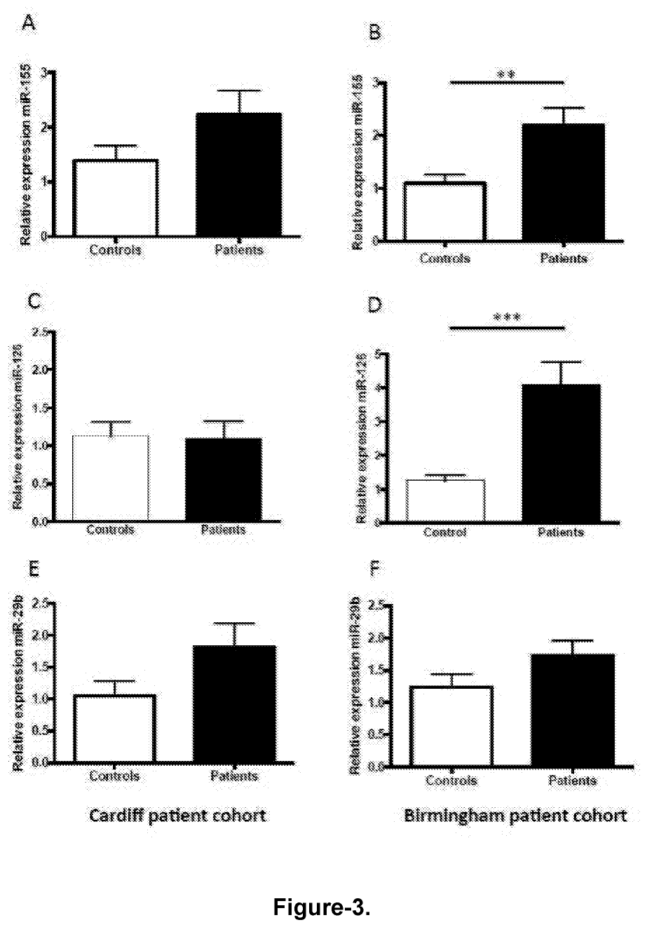

FIG. 3: Validation of significantly increased detection of selected microRNAs in independent diabetic nephropathy cohorts. RT-qPCR analysis shows an absence of significant difference in detection of (A) hsa-miR-155-5p, (C) hsa-miR-126-3p and (E) hsa-miR-29b-3p between patients and controls in an independent patient cohort from Cardiff (99 patients, 82% either CKD stages 1 or 2; 20 control subjects). RT-qPCR analysis shows significant differences in detection of (B) hsa-miR-155-5p (p=0.003) and (C) hsa-miR-126-3p (p=0.001), with a trend toward significance for (E) hsa-miR-29b-3p between patients and controls in an independent patient cohort from Birmingham (70 patients, 97% CKD stages 3-5; 22 control subjects). Analysis was carried out by unpaired t-test with Welch's correction. Data were normalized to endogenous control hsa-miR-191-5p and are presented as mean+/−SEM;

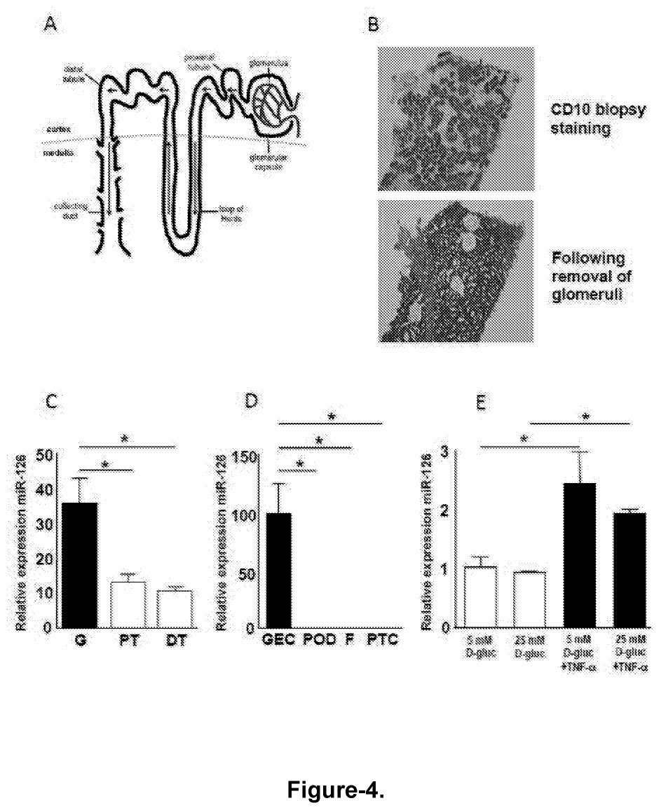

FIG. 4: Localization of hsa-miR-126-3p expression by laser capture microdissection (LCM). (A) The nephron spans the renal cortex and medulla and includes the glomerulus, the proximal tubule and the distal tubule, all key functional domains. (B) LCM was used to excise discrete glomeruli from a CD10-stained FFPE renal biopsy sample. (C) Relative expression of hsa-miR-126-3p in LCM-isolated glomeruli (G), proximal tubules (PT) and distal tubules (DT) from 5 renal biopsies of healthy individuals. (D) Relative expression of hsa-miR-126-3p in in vitro cultured glomerular endothelial cells (GEC), podocytes (P), fibroblasts (F) and proximal tubular cells (PTC). (E) Relative expression of hsa-miR-126-3p in glomerular endothelial cells following 24 h culture in 5 mM (normoglycaemic) or 25 mM (hyperglycaemic) D-glucose (D-gluc)+/−10 ng/ml TNF-α. Analysis was carried out by one-way ANOVA analysis with Tukey's multiple comparison test and, where appropriate, data are presented as mean+/−SEM. *, p 0.05;

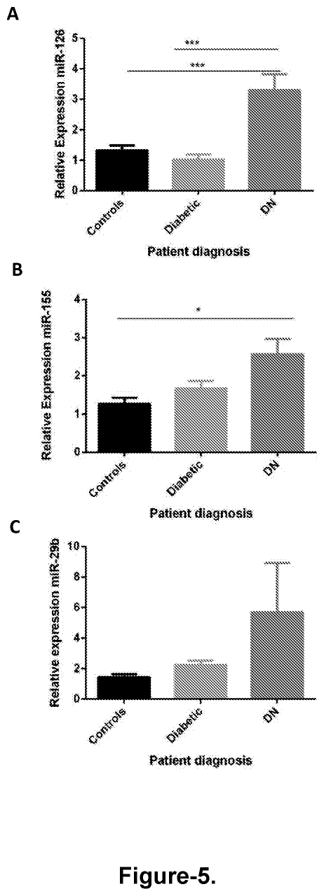

FIG. 5: Analysis of selected miRNAs in diabetic nephropathy (DN) patients, diabetic patients and controls. RT-qPCR analysis shows (A) no significant difference in detection of hsa-miR-126-3p between diabetic patients and controls, and a significant difference between both these groups and diabetic nephropathy patients (p=0.0221), (B) hsa-miR-155-5p is significantly different when comparing both diabetic patients and controls with diabetic nephropaths (p=0.0024) and (C) hsa-miR-29b-3p trends toward significance in diabetic nephropathy patients. Analysis was carried out by one-way ANOVA with Tukey's test for multiple comparison. Data were normalized to endogenous control miR-191 and are presented as mean+/−SEM;

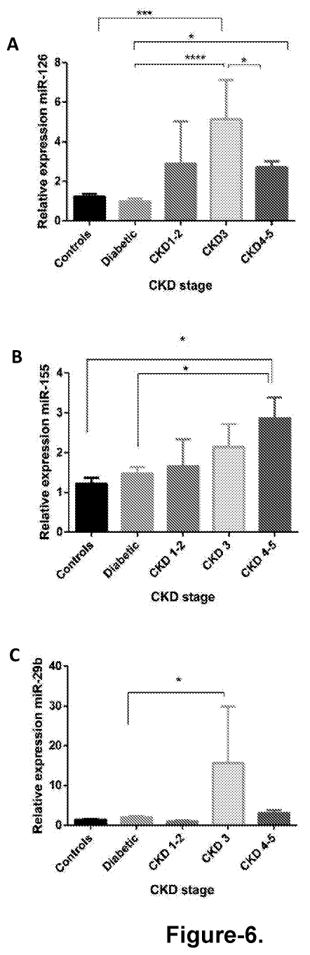

FIG. 6: Analysis of selected miRNAs in controls, diabetic patients and diabetic nephropathy patients categorized by CKD stage. RT-qPCR analysis shows a significant difference in detection of (A) hsa-miR-126-3p between control and diabetic groups in comparison with diabetic nephropathy patients at CKD stages 3-5 (p=0.0011; 0.046; 0.0001 and 0.038), (B) hsa-miR-155-5p is significantly different when comparing controls and diabetic patients with diabetic nephropathy patients at CKD stages 4-5 (p=0.024 and 0.043) and (C) hsa-miR-29b-3p is significantly different when comparing diabetic patients with diabetic nephropathy patients at CKD stage 3 (p=0.028). Analysis was carried out by one-way ANOVA with Tukey's test for multiple comparison. Data were normalized to endogenous control miR-191 and are presented as mean+/−SEM;

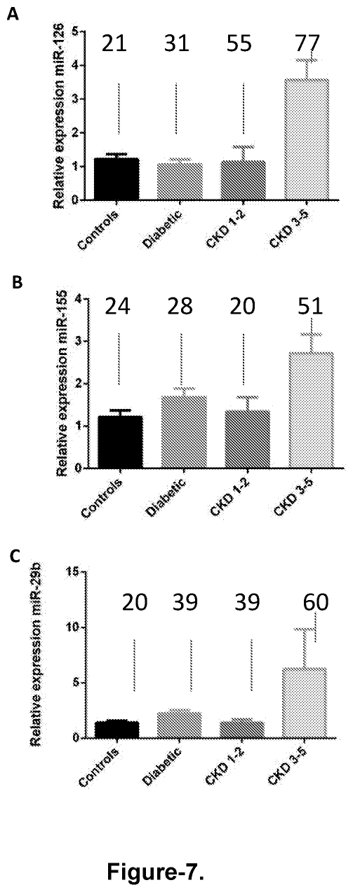

FIG. 7: Analysis of >2-fold increased detection of selected miRNAs in controls, diabetic patients and diabetic nephropathy patients categorized by CKD stage. RT-qPCR analysis shows increased percentages of diabetic nephropathy patients at CKD stage 3-5 detection with a >2-fold increased detection of (A) hsa-miR-126-3p, (B) hsa-miR-155-5p and (C) hsa-miR-29b-3p compared to controls, diabetic patients and diabetic nephropathy patients at CKD stages 1-2;

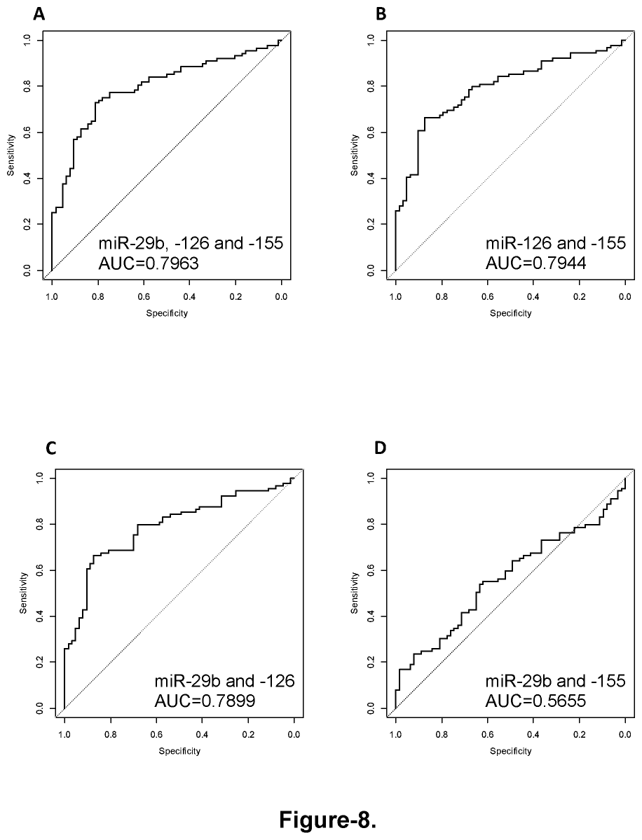

FIG. 8: Combined receiver operating characteristic (ROC) curve analysis comparing diabetic patients and diabetic nephropathy patients. Combined ROC curve analysis comparing (A) hsa-miR-155-5p, hsa-miR-126-3p and hsa-miR-29b-3p for which the area under the curve (AUC)=0.7963, (B) hsa-miR-155-5p and hsa-miR-126-3p (AUC=0.7944), (C) hsa-miR-126-3p and hsa-miR-29b-3p (AUC=0.7899) and (D) hsa-miR-155-5p and hsa-miR-29b-3p (AUC=0.5655). ROC curves were generated and AUC values computed using the pROC package in R-3.2.3; and

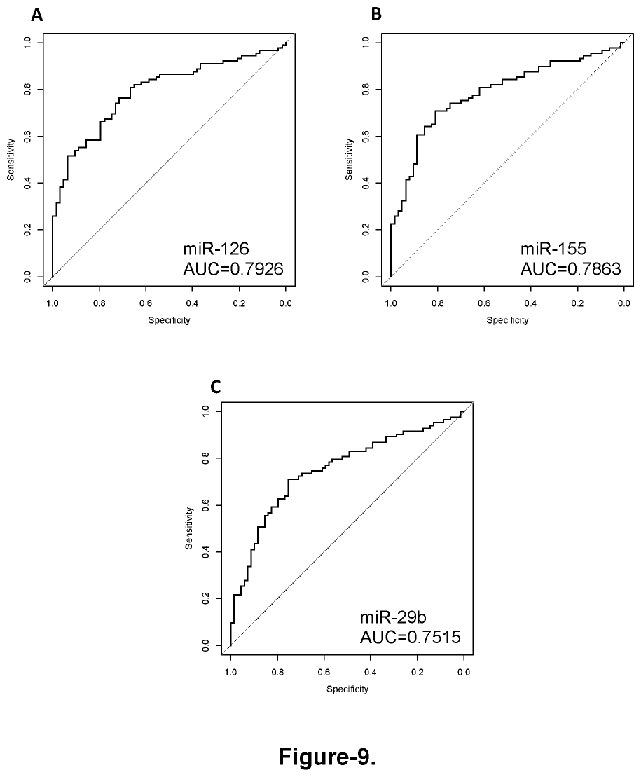

FIG. 9: Individual ROC curve analysis comparing diabetic patients and diabetic nephropathy patients. Individual ROC curve analysis for (A) hsa-miR-126-3p (AUC=0.7926), (B) hsa-miR-155-5p (AUC=0.7863) and (C) hsa-miR-29b-3p (AUC=0.7515). ROC curves were generated and AUC values computed using the pROC package in R-3.2.3.

Table 1: Independent diabetic nephropathy patient cohort details.

Table 2: Independent diabetic nephropathy patient and diabetes patient cohort details. This is a revised analysis of the data presented in Table 1.

Table 3: Specificity values and likelihood ratios for hsa-miR-155-5p, hsa-miR-126-3p, hsa-miR-29b-3p and all three miRNAs above a ROC curve sensitivity threshold of 80%.

Table 4: Diabetic patient and diabetic nephropathy patient numbers and percentages above a ROC curve sensitivity threshold of 80% for hsa-miR-155-5p, hsa-miR-126-3p, hsa-miR-29b-3p and all three miRNAs.

### Materials and Methods

### Study Population

Urine samples were obtained from the Wales Kidney Research Tissue Bank and approval for the study granted by its governance committee. Details of patient samples are outlined in table 1.

### Urine Collection and RNA Isolation

Second pass morning urine (20 mL) was collected, placed on ice and processed immediately. Samples were centrifuged at 2000 g for 10 min at 4° C., and supernatants transferred to fresh, sterile universal tubes for RNA isolation. RNA was isolated from all samples using the miRNeasy kit (Qiagen, Crawley, West Sussex, UK) with the following modifications to the manufacturer's protocol.

### Reverse Transcription-Quantitative PCR (RT-qPCR) Analysis

Reverse transcription (RT) was carried out using the High-Capacity cDNA RT Kit (4368814, Life Technologies, Carlsbad, Mass., USA). The RT master mix for one reaction was composed of: 4.25 μL of water, 1.5 μL of 10×RT Buffer, 0.15 μL of 100 mM dNTP, 0.1 μL of 40 U/μL RNase Inhibitor (M0307S, New England BioLabs® Inc., Ipswich, Mass., USA), 1 μL of 50 U/μL MultiScribe Reverse Transcriptase and 3 μL of 5×RT-primer specific for each miRNA. Then, 10 μL of the RT master mix was added to 4 μL of water plus 1 μL of RNA for the urine samples. The RT non-template control (RT-NTC) negative control reaction contained an equal volume of water instead of RNA. The following thermal cycler profile was used: 30 min at 16° C., 30 min at 42° C., 5 min at 85° C., followed by cooling to 4° C. The cDNA was diluted with water 1:3, and 4 μL was used in qPCR. For each transcript, the master mix for each reaction was prepared by combining 1 μL of miRNA-specific set of PCR-primers and TaqMan probe (Life Technologies), 5 μL of water and 2×Universal PCR Master Mix (4440047, Life Technologies). MiRNA-specific master mix (16 μL) was distributed to appropriate wells on an Optical 96-Well Fast Plate (Life Technologies) followed by 4 μL of pre-diluted cDNA, or water for NTCs. Plates were sealed with MicroAmp Optical Adhesive Film (Life Technologies) and qPCR was performed on a ViiA7 Real-Time PCR System (Life Technologies) using the manufacturer's recommended cycling parameters: 10 min at 95° C. followed by 40 cycles of 15 s at 95° C. and 1 min at 60° C. Relative expression was calculated by standard means.

Specific miRNA oligonucleotide kits were obtained from Thermofisher Scientific, using the following assay ID numbers: hsa-miR-191-5p (#002299); hsa-miR-29b-3p (#000413); hsa-miR-126-3p (#002228); and hsa-miR-155-5p (#002623).

### Laser Capture Microdissection (LCM)

LCM was performed using the The Arcturus PixCell® system.

Tissue preparation—Coverslip-free H&E stained sections of tissue on non-charged uncoated microscope slides (VFM slides CellPath ltd) were prepared for LCM. Contiguous sections were prepared on charged (Flex™ slides, Dako) and stained for CD10 to enable precise identification of nephron segments. In both cases, 6 micron sections were used. Tissue preparation was performed by the department of cellular pathology, University Hospital of Wales. The detailed staining methods are found below.

H&E staining

| | | |
|---|---|---|
| 1. | Cut 6 um sections onto plain uncoated | 1 hour |
| | slides and dry at 60° C. | |
| 2. | Xylene | 1 min |
| 3. | Xylene | 1 min |
| 4. | Xylene | 1 min |
| 5. | Industrial denatured alcohol | 1 min |
| 6. | Industrial denatured alcohol | 1 min |
| 7. | Industrial denatured alcohol | 1 min |
| 8. | Running tap water | 30 sec |
| 9. | Mayer's Haematoxylin | 3 min |
| 10. | Warm running tap water | 1 min |
| 11. | 1% Eosin | 1 min |
| 12. | Running tap water | 30 sec |
| 13. | Industrial denatured alcohol | 1 min |
| 14. | Industrial denatured alcohol | 1 min |
| 15. | Industrial denatured alcohol | 1 min |
| 16. | Allow to air dry | |

CD10 staining was performed using the EnVision Flex™ system from Dako.

The protocol in brief is as follows.

| | | |
|---|---|---|
| 1. | Cut 4 um sections onto Flex ™ slides and dry at 60° C. | 1 hour |
| 2. | Place slides in Dako PT module chamber | 20 min |
| 3. | Dako wash buffer | 5 min |
| 4. | Load slide into Dako Autostainer Link 48 ™ staining | |
| | machine | |
| | Wash buffer rinses ×2 | |
| | Primary antibody (NCL-L-CD10-270) | 20 min |
| | Wash buffer rinse | |
| | Dako Flex ™ mouse linker | 15 min |
| | Wash buffer rinse | |
| | Flex/HRP polymer | 20 min |
| | Wash buffer rinses ×2 | |
| | Substrate working solution | 5 min |
| | Substrate working solution | 5 min |
| | Wash buffer rinse | |
| | Counterstain with Dako Flex ™ Haematoxylin | 5 min |
| | Distilled water rinse | |

Slides are then removed and dehydrated with repeated ethanol washes and ‘cleared’ through a series of xylene washes before being cover slipped.

RNA Extraction—RNA extraction was performed using the RecoverAll Total Nucleic Acid Isolation Kit (Ambion), according to the manufacturer's instructions for isolation of RNA from formalin fixed paraffin embedded (FFPE) tissue samples. The polymer membrane from the LCM cap was first removed using clean tweezers and placed in an Eppendorf. Digestion buffer and protease were then added and samples incubated at 50° C. for 15 minutes then 15 minutes at 80° C. Subsequently, isolation additive/ethanol mixture was added to the samples and mixed by repeated pipetting. This mixture was then placed in a filter cartridge in a fresh collection tube and centrifuged at 10000 rcf for 30 seconds to pass the mixture through the filter. The flow-through was then discarded. The filters were then washed with wash 1 and wash ⅔ buffer, each time centrifuging to pass the mixture through the filter and discarding the flow-through. DNA was then removed from the filter by incubating with DNase mix at room temperature for 30 minutes. Again the filter was washed once with wash 1 and twice with wash ⅔ before 60 ul of elution solution was added to the center of the filter. The filter was then placed in a fresh collection tube and the mixture centrifuged. 1 ul aliquots were then taken for quality control and the residual sample stored at −80° C.

### Statistical Data

RT-qPCR data for individual miRNAs were analysed using GraphPad Prism version 6 software to run the student's t-test. Values for p below 0.05 were considered significant. Receiver operating characteristic ROC curve analysis for individual miRNAs and combinations thereof was carried out using the pROC package in R-3.2.3 and the ROC function in GraphPad Prism version 6. Clinical data were analysed and probabilities computed using Microsoft Excel.

### Results

FIG. 1 shows a volcano plot comparing microRNA (miRNA) detection in pooled urine samples from late-stage diabetic nephropathy patients (CKD stages 3-5) compared with unaffected individuals generated using the TaqMan Low Density Array (TLDA) system.

Pool 1 control=5 female urine samples; average age=44.8
Pool 2 control=5 female urine samples; average age=57.6
Pool 3 control=5 male urine samples; average age=35.2
Pool 4 control=5 male urine samples; average age=53.2
Pool 1 patient=stage 3 and eGFR between 43.3 and 36 mL/min per 1.73 m²
Pool 2 patient=stage 3 and eGFR between 35 and 31 mL/min per 1.73 m²
Pool 3 patient=stage 4/5 and eGFR between 27.3 and 23 mL/min per 1.73 m²
Pool 4 patient=stage 4/5 and eGFR between 22 and 12.9 mL/min per 1.73 m²

Each point on the plot depicts one miRNA, and the red points in the upper right section of the plot represent miRNAs with statistically significantly increased detection in patients compared to controls, while the green points in the upper left section represent miRNAs with significantly decreased detection in patients compared to controls. These data are depicted in histogram form in Panel B.

FIG. 2 shows RT-qPCR detection for hsa-miR-155-5p, hsa-miR-126-3p and hsa-miR-29b-3p, normalised relative to hsa-miR-191-5p, in the individual urine samples making up the pools that were analysed in FIG. 1 (see above for pool composition details). These data replicated the significant differences found in the pooled samples, and were generated using the ThermoFisher Scientific miRNA-specific assays in the individual urine samples making up the pools that were analysed in FIG. 1 (see above for pool composition details). Receiver operating characteristic (ROC) curves calculated using these detection data are also shown.

FIG. 3 shows data from the same RT-qPCR analysis methods described for FIG. 2 for hsa-miR-155-5p, hsa-miR-126-3p and hsa-miR-29b-3p in independent validation cohorts from Cardiff and Birmingham. Further details of the patients and control subjects are provided in Table 1.

FIG. 4 shows data from laser capture microdissection from biopsies of unaffected individuals. Panel A depicts the nephron in diagrammatic form, and Panel B shows a typical biopsy before and after laser microdissection of glomeruli. In panel C, the detection of hsa-miR-126-3p is shown in glomerular, proximal tubular and renal tubular microdissected samples, and reveals that detection of this transcript is significantly increased in the glomerulus. Panel D provides data for glomerular endothelial and podocyte cell lines derived from the glomerulus, as well as fibroblast and proximal tubular cells, and shows hsa-miR-126-3p expression is detected primarily in glomerular endothelial cells. Panel E shows the significant increase in hsa-miR-126-3p detection in glomerular endothelial cell culture medium following treatment with 10 ng/ml tumour necrosis factor (TNF)-alpha and either 5 mM or 25 mM D-glucose.

FIG. 5 shows data from the same RT-qPCR analysis methods described for FIG. 2 for hsa-miR-155-5p, hsa-miR-126-3p and hsa-miR-29b-3p. The data shown were obtained from the independent validation cohorts from Cardiff and Birmingham described for FIG. 3 and detailed in Table 1. For FIG. 5, these data were reanalysed as one validation cohort sample, the details of which are provided in Table 2. Control subjects, diabetic patients and diabetic nephropaths were analysed as discrete groups, and the differences in miRNA detection seen previously were again observed.

FIGS. 6 and 7 show analysis of the Table 2 patients described above with diabetic nephropathy patients divided on the basis of their disease stage. In FIG. 6, significant differences were observed in CKD stages 3-5, and FIG. 7 shows that the highest percentage of these patients had a greater than 2-fold increase in target miRNA detection.

FIGS. 8 and 9 show ROC curves calculated from the RT-qPCR relative expression (RQ) data for hsa-miR-155-5p, hsa-miR-126-3p and hsa-miR-29b-3p, separately or in combination, from the Cardiff and Birmingham patient cohorts described above. Further analysis of these data in Table 3 shows that for a sensitivity (true positive rate) of approximately 80%, the increase in specificity (100—false positive rate) using data from all three miRNAs was more than 10% when compared to individual miRNAs. Table 4 shows that when this threshold was applied successively for each miRNA, a true positive rate of approximately 48% and a false positive rate of less than 4% were observed.

TABLE 1

| | Cardiff | | Birmingham | |
|---|---|---|---|---|
| | early DN Cohort | | advanced DN Cohort | |
| | Patients n = 99 | Controls n = 20 | Patients n = 70 | Controls n = 22 |
| Male n (%) | 59 (59.6) | 8 (40) | 43 (61.4) | 10 (45.5) |
| Non-Caucasian n (%) | 18 (18.2) | | 28 (40) | |
| Mean Age (SD) | 55 +/− 16.5 | 41.4 +/− 10.6 | 61.6 +/− 13.58 | 67 +/− 4.9 |
| eGFR | | | | |
| Mean (SD) | 74.7 +/− 19.5 | | 23.4 +/− 14.7 | |
| Median (IQR) | 82.5 (68.5-90) | | 19.5 (15.8-25.1) | |
| CKD stage n (%) | | | | |
| No CKD/CKD 1 | 35 (35.7) | | 2 (2.9) | |
| CKD 2 | 45 (45.5) | | 0 | |
| CKD 3 | 13 (13.1) | | 10 (14.3) | |
| CKD 4 | 5 (5.1) | | 43 (61.4) | |
| CKD 5 | 0 | | 15 (21.4) | |
| Proteinuria | | | | |
| Absent (ACR < 30, | 77 (77.8) | | 23 (32.9) | |
| PCR < 20) | | | | |
| Micro (ACR 30-300, PCR | 20 (20.2) | | 33 (47.1) | |
| 20-300) | | | | |
| Macro (ACR/PCR > 300) | 2 (2) | | 14 (20) | |

TABLE 2

| | Patients (n = 151) | | Control (n = 41) |
|---|---|---|---|
| | | Diabetic | Control |
| | Diabetes (n = 62) | Nephropathy (n = 89) | Subjects |
| Male n (%) | 37 (58) | 55 (62) | 18 (44) |
| Non-Caucasian n (%) | 13 (21) | 33 (37) | |
| Mean Age (SD) | 52 +/− 16.1 years | 62 +/− 13.3 years | 55 +/− 15.4 years |
| eGFR mls/min/1.73 m2 | | | |
| Mean (SD) | 78 +/− 16.3 | 30 +/− 20.9 | |
| Median (IQR) | 84 (72-90) | 22 (17-38) | |
| CKD stage n (%) | | | |
| No CKD/CKD 1 | 23 (37) | 2 (2) | |
| CKD 2 | 32 (52) | 10 (11) | |
| CKD 3 | 5 (8) | 17 (19) | |
| CKD 4 | 2 (3) | 45 (51) | |
| CKD 5 | 0 | 15 (17) | |
| Albumin:creatinine ratio | | | |
| (ACR) mg/mmol n (%) | | | |
| Normal-high normal (ACR < 3) | 54 (87.1) | 15 (16.9) | |
| Moderately increased (ACR 3-30) | 8 (12.9) | 25 (28.1) | |
| Severely increased (ACR > 30) | 0 (0) | 49 (55.0) | |

TABLE 3

| | Sensitivity | | | |
|---|---|---|---|---|
| | Threshold | Specificity | Likelihood | RQ |
| | (%) | (%) | Ratio | Threshold |
| All | 80.21 | 63.64 | 2.206 | >1.148 |
| miRNAs | | | | |
| miR-126 | 80.41 | 57.14 | 1.876 | >0.6762 |
| miR-155 | 80.61 | 52 | 1.679 | >0.9110 |
| miR-29b | 80.61 | 40 | 1.344 | >0.8058 |

TABLE 4

| | | | | |
|---|---|---|---|---|
| miR-126 | miR-155 | miR-29b | All miRNAs | Total patients |

Patients above 80% sensitivity threshold

| | | | | | |
|---|---|---|---|---|---|
| D | 23 | 30 | 13 | 2 | 55 |
| DN | 80 | 67 | 73 | 47 | 98 |

Percentage of patients above 80% sensitivity threshold

| | | | | |
|---|---|---|---|---|
| D | 41.8 | 54.6 | 23.6 | 3.6 |
| DN | 81.6 | 68.4 | 74.5 | 48.0 |

1. A diagnostic method for determining chronic kidney disease in a human subject comprising detecting the level(s) or amount(s) of at least one of the miRNAs selected from the group consisting of: hsa-miR-126-3p, hsa-miR-155-5p, and hsa-miR-29b-3p, or any combination thereof, in an isolated urine sample from said human subject wherein an increased level(s) or amount(s) of said at least one miRNA(s) relative to the level(s) or amount(s) of the same miRNA, or combination thereof, in a control is indicative of chronic kidney disease.

2. The method according to claim 1 wherein said chronic kidney disease is an advanced stage (any one of iii-v) of chronic kidney disease.

3. The method according to claim 1 wherein said method comprises the detection of hsa-miR-126-3p and/or hsa-miR-155-5p and/or hsa-miR-29b-3p mRNA, or their respective cDNA.

4. The method according to claim 1 wherein said method comprises:
i) providing a urine sample taken from a subject to be analyzed;
ii) forming a reaction mixture comprising the urine sample and one or more nucleic acid probes complementary to all or a part of a nucleotide encoding at least one of, or any combination of, hsa-miR-126-3p, hsa-miR-155-5p, and hsa-miR-29b-3p;
iii) detecting and/or amplifying a nucleic acid molecule encoding at least one of, or any combination of hsa-miR-126-3p, hsa-miR-155-5p, and hsa-miR-29b-3p in said urine sample;
iv) quantifying the level or amount of at least one of, or any combination of, hsa-miR-126-3p, hsa-miR-155-5p, and hsa-miR-29b-3p in said urine sample and comparing the said level or amount to a level or amount of the same miRNA, or same combination of miRNAs, in a control; wherein an increase in the level or amount of at least one or any combination of hsa-miR-126-3p, miR-155-5p, and hsa-miR-29b-3p in said urine sample relative to said same miRNA, or same combination thereof, in said control indicates chronic kidney disease in said subject.

5. The method according to claim 1 wherein said method detects the level or amount of one or more of hsa-miR-126-3p, hsa-miR-155-5p and hsa-miR-29b-3p mRNA, or their respective cDNA.

6. The method according to claim 1 wherein the urine sample is a processed derivative of a urine sample.

7. The method according to claim 1 wherein said urine sample comprises one or more of cells, exosomes and vesicles.

8. The method according to claim 1 wherein hsa-miR-126-3p, hsa-miR-155-5p, and hsa-miR-29b-3p comprise the nucleotide sequence set forth in SEQ ID NO: 1-3, respectively.

9. The method according to claim 1 wherein said method is a PCR based method.

10. The method according to claim 9 wherein said PCR based method is Real Time [RT] PCR.

11. The method according to claim 9 wherein detection is achieved using specific nucleic acid oligonucleotide probes that can detect the level or amount of said miRNA(s).

12. The method according to claim 11 wherein said probes are adapted to quantify the level or amount of said miRNA(s).

13. The method according to claim 11 wherein said oligonucleotide probes comprise at least 7 consecutive bases which are complementary to a target miRNA sequence.

14. The method according to claim 11 wherein said oligonucleotide probe(s) are designed to bind to the nucleotide sequences set forth in SEQ ID NOs: 1-3.

15. The method according to claim 1 wherein the level or amount of expression of a given miRNA is normalised to the expression of a reference miRNA.

16. The method according to claim 15 wherein said reference miRNA is hsa-miR-191-5p.

17. The method according to claim 1 wherein the level or amount of at least one of hsa-miR-126-3p, hsa-miR-155-5p, and hsa-miR-29b-3p is determined having regard to a reference gene or protein within a control sample or from the same sample.

18. The method according to claim 1 wherein the level or amount of at least one of hsa-miR-126-3p, hsa-miR-155-5p, and hsa-miR-29b-3p is compared to an internal standard.

19. The method according to claim 1 wherein at least one of hsa-miR-126-3p, hsa-miR-155-5p, and hsa-miR-29b-3p is at least 1.5-fold higher relative to the level or amount of same in a control sample taken from a healthy individual or an individual not having or presenting with CKD or late stage (iii-v) CKD.

20. The method according to claim 19 wherein at least one of hsa-miR-126-3p, hsa-miR-155-5p, and hsa-miR-29b-3p is determined to be present in a level or amount that is 1.5-6.0-fold higher relative to the level or amount of same in said control sample.

21. The method according to claim 20 wherein at least one of hsa-miR-126-3p, hsa-miR-155-5p, and hsa-miR-29b-3p is of an amount selected from the group comprising: 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, and 6.0 fold higher relative to the level or amount of same in said control sample.

22. A method for monitoring the progression of chronic kidney disease in a human subject comprising repeating the method according to claim 1 periodically.

23. A method for selecting a course of treatment for treating chronic kidney disease comprising performing the method according to claim 1 and then, depending upon the outcome of the method, determining a suitable or selected course of treatment.

24. A method for treating chronic kidney disease comprising performing the method according to claim 1 and then, depending upon the outcome of the method, determining a suitable or selected course of treatment.

25. A kit for performing the method according to claim 1 wherein said kit comprises:
(a) at least one oligonucleotide probe adapted for detecting and/or quantifying a level or amount of at least one of hsa-miR-126-3p, hsa-miR-155-5p, and hsa-miR-29b-3p in a urine sample; and
(b) optionally, reagents and instructions pertaining to the use of said oligonucleotides.

26. The kit according to claim 25 wherein the at least one oligonucleotide probe is designed to bind to a nucleotide sequence set forth in at least one of SEQ ID NOs: 1-3.

27. The kit according to claim 25 wherein said kit comprises a plurality of said oligonucleotide probes.

28. The kit according to claim 27 wherein the plurality of said oligonucleotides probes includes probes designed to bind to a plurality of nucleotide sequences set forth in SEQ ID NOs: 1-3.

29. The kit according to claim 27 wherein the plurality of said oligonucleotides probes includes probes designed to bind to all the nucleotide sequences set forth in SEQ ID NOs: 1-3.

30. The kit according to claim 25 further comprising at least one oligonucleotide probe(s) for detecting and quantifying a reference miRNA.

31. The kit according to claim 30 wherein said reference miRNA is hsa-miR-191-5p.
